(19) United States
(12) Patent Application Publication
(10) Pub. No.: US 2008/0139584 A1
(43) Pub. Date: Jun. 12, 2008

(54) Method for healing a wound

(76) Inventors: Matthew H. Kopacki, Ringwood, NJ (US); Michael J. Torsiello, Pleasantmount, PA (US)

Correspondence Address: King & Partners, PLC, 170 College Avenue, SUITE 230, HOLLAND, MI, 49423, US

(21) Appl. No.: 11/978,293

(22) Filed: Oct. 29, 2007

Related U.S. Application Data
(60) Provisional application No. 60/854,805, filed on Oct. 27, 2006.

(51) Int. Cl. A61K 31/52; A61K 31/44; A61P 17/02

(52) U.S. Cl. 514/263.32; 514/354

(57) ABSTRACT

A method for healing a wound comprising the steps of: topically administering a wound healing composition to a wounded area, wherein the wound healing composition comprises: a first medicament characterized as a calcium channel blocker or pharmaceutically acceptable salts or solvates thereof; a second medicament characterized as a hemorrheologic agent or pharmaceutically acceptable salts or solvates thereof; and a dermal penetrating agent or pharmaceutically acceptable salts or solvates thereof.

### CROSS REFERENCE TO RELATED APPLICATION(S)

This application claims the benefit of U.S. Provisional Application Ser. No. 60/854,805, filed Oct. 27, 2006, entitled “METHOD FOR HEALING A WOUND,” which is hereby incorporated herein by reference in its entirety, including all references cited therein.

### BACKGROUND OF THE INVENTION

1. Field of the Invention

The present invention relates in general to a method for healing a wound and, more particularly, to a method for healing a wound via localized, topical application of a first medicament, a second medicament, and a dermal penetrating agent.

2. Background Art

In today's society, many people suffer from difficult-to-heal wounds that are refractory to conventional forms of treatment. Such difficult-to-heal wounds may include diabetic skin sores, pressure sores, vessel disease wounds, surgery wound breakdown, spinal injury wounds, and chemical wounds—just to name a few.

To be sure, the human body's healing process is very complex and requires several steps. According to The Wound Care Information Center, sponsored by the Wound Care Center at Presbyterian Hospital of Dallas, normal healing requires that cells proliferate and divide, thereby releasing growth factors. In turn, new blood vessels are produced, a collagen matrix is formed, and remodeling occurs. Each step requires appropriate substrates and nutritional elements to be present and available. However, for some patients, certain conditions alter this course, thereby disrupting the healing process. In such cases, the wound can become chronic. In America alone, approximately five million (5,000,000) people are battling chronic open sores which may become seriously infected, gangrenous, and may eventually require amputation.

In addition, the Wound Care Information Center estimates that there are approximately sixteen million (16,000,000) diabetics in America. Diabetes causes microangiopathic changes in, among other places, the foot, which is a common site for non-healing wounds. It is not unlikely for diabetic patients to have to undergo surgical amputation as a result of a non-healing wound and must then face a lifetime of costly rehabilitation, and permanently reduced mobility and independence.

Other conditions which can lead to the development of non-healing wounds include peripheral vascular disease, arterial or venous ulcers, traumatic injury, complications following surgery, rheumatoid arthritis, congestive heart failure, lymphedema, and other conditions which compromise circulation. In addition, local factors such as pressure, infection, or edema, and systemic problems which leave patients immunocompromised, such as collagen vascular disease, acquired immunodeficiency syndrome, rheumatoid arthritis, or diabetes mellitus, can impair normal healing. Furthermore, some medications can suppress the body's healing process and inadequate large-vessel perfusion and oxygenation impedes healing by reducing the oxygen supply to the damaged tissue.

At this time, wound treatment programs and intervention include conventional and advanced wound dressings, removal of unhealthy tissue, bioengineered tissue, hyperbaric (high-pressure) oxygen treatment, growth factors (isolated, concentrated substances that are applied topically to the wound to stimulate healing), antibiotic therapy, nutrition counseling, education and prevention, surgery, physical therapy, and protective footwear, among others.

Patients suffering from such wounds and/or chronic open sores typically seek specialized professional help after their wounds have not healed during months of standard wound treatment. However, even the most advanced methods for healing wounds can take several additional months and are not always successful.

It is therefore an object of the present invention, to provide a method for healing a wound via localized, topical application of a first medicament, a second medicament, and a dermal penetrating agent to remedy and/or minimize the aforementioned problems and/or complications associated with healing a wound.

### SUMMARY OF THE INVENTION

The present invention is directed to a method for healing a wound comprising the steps of topically administering a wound healing composition to a wounded area, wherein the wound healing composition comprises: (a) a first medicament characterized as a calcium channel blocker or pharmaceutically acceptable salts or solvates thereof; (b) a second medicament characterized as a hemorrheologic agent or pharmaceutically acceptable salts or solvates thereof; and (c) a dermal penetrating agent or pharmaceutically acceptable salts or solvates thereof.

In a preferred embodiment of the present invention, the first medicament is represented by the following chemical structure:

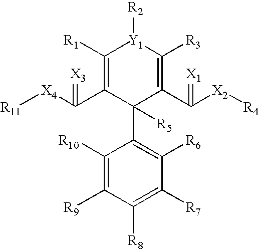

wherein R₁₋₁₁ are the same or different and comprise H, a hydroxy group, a primary, secondary, or tertiary amine, a halide, NO₂, SO₃H, CN, a straight or branched alkyl, cycloalkyl, polycycloalkyl, heterocycloalkyl, aryl, alkaryl, aralkyl, alkoxy, alkenyl, alkynyl, or carbonyl group containing approximately 1 to approximately 50 carbon atom(s), a silyl or siloxyl group containing approximately 1 to approximately 50 silicon atom(s), and combinations thereof, preferably with the proviso that at least one of R₆-R₁₀ comprises a halide, NO₂, SO₃H, or CN; wherein X₁₋₄ are the same or different and comprise oxygen, sulfur, or selenium; and wherein Y₁ comprises nitrogen or phosphorus.

In another preferred embodiment of the present invention, the first medicament is represented by the following chemical structure:

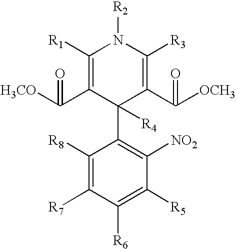

wherein R₁₋₈ are the same or different and comprise H, a hydroxy group, a primary, secondary, or tertiary amine, a straight or branched alkyl, cycloalkyl, polycycloalkyl, heterocycloalkyl, aryl, alkaryl, aralkyl, alkoxy, alkenyl, alkynyl, or carbonyl group containing approximately 1 to approximately 50 carbon atom(s), a silyl or siloxyl group containing approximately 1 to approximately 50 silicon atom(s), and combinations thereof. In this embodiment, the first medicament may be represented by the following chemical structure:

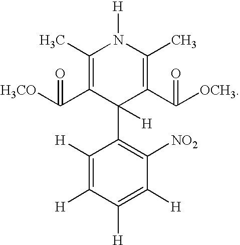

In yet another preferred embodiment of the present invention, the second medicament is represented by the following chemical structure:

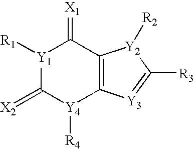

wherein R₁₋₄ are the same or different and comprise H, a hydroxy group, a primary, secondary, or tertiary amine, a straight or branched alkyl, cycloalkyl, polycycloalkyl, heterocycloalkyl, aryl, alkaryl, aralkyl, alkoxy, alkenyl, alkynyl, or carbonyl group containing approximately 1 to approximately 50 carbon atom(s), a silyl or siloxyl group containing approximately 1 to approximately 50 silicon atom(s), and combinations thereof; wherein X₁₋₂ are the same or different and comprise oxygen, sulfur, or selenium; and wherein Y₁₋₄ are the same or different and comprise nitrogen or phosphorus.

In another aspect of the present invention, the second medicament is represented by the following chemical structure:

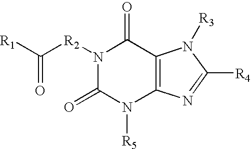

wherein R₁₋₅ are the same or different and comprise H, a hydroxy group, a primary, secondary, or tertiary amine, a straight or branched alkyl, cycloalkyl, polycycloalkyl, heterocycloalkyl, aryl, alkaryl, aralkyl, alkoxy, alkenyl, alkynyl, or carbonyl group containing approximately 1 to approximately 50 carbon atom(s), a silyl or siloxyl group containing approximately 1 to approximately 50 silicon atom(s), and combinations thereof. In this embodiment, the second medicament may be represented by the following chemical structure:

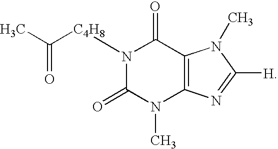

In a preferred embodiment of the present invention, the dermal penetrating agent is represented by the following chemical structure:

R₁—X₁—R₂—X₂—R₃—X₃—R₄

wherein R₁₋₄ are the same or different and comprise H, a hydroxy group, a primary, secondary, or tertiary amine, a straight or branched alkyl, cycloalkyl, polycycloalkyl, heterocycloalkyl, aryl, alkaryl, aralkyl, alkoxy, alkenyl, alkynyl, or carbonyl group containing approximately 1 to approximately 50 carbon atom(s), a silyl or siloxyl group containing approximately 1 to approximately 50 silicon atom(s), and combinations thereof; and wherein X₁₋₃ are the same or different and comprise oxygen, sulfur, or selenium.

In another preferred embodiment of the present invention, the dermal penetrating agent is represented by the following chemical structure:

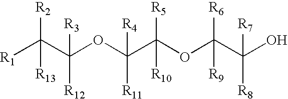

wherein R₁₋₁₃ are the same or different and comprise H, a hydroxy group, a primary, secondary, or tertiary amine, a straight or branched alkyl, cycloalkyl, polycycloalkyl, heterocycloalkyl, aryl, alkaryl, aralkyl, alkoxy, alkenyl, alkynyl, or carbonyl group containing approximately 1 to approximately 50 carbon atom(s), a silyl or siloxyl group containing approximately 1 to approximately 50 silicon atom(s), and combinations thereof. In this embodiment, the dermal penetrating agent may be represented by the following chemical structure:

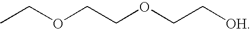

The present invention is also directed to topically administering a wound healing composition which comprises a first medicament comprising 3,5-pyridinedicarboxylic acid, 1,4-dihydro-2,6-dimethyl-4-(2-nitrophenyl)-dimethyl ester and pharmaceutically acceptable salts or solvates thereof; a second medicament comprising 1-(5-oxohexyl)-3,7-dimethylxanthine and pharmaceutically acceptable salts or solvates thereof; and a dermal penetrating agent comprising 2-(2-ethoxy-ethoxy)-ethanol and pharmaceutically acceptable salts or solvates thereof.

In a preferred embodiment of the present invention, an effective amount of a wound healing composition as provided herein is topically administered to a wounded area.

### DETAILED DESCRIPTION OF THE INVENTION

While this invention is susceptible of embodiment in many different forms, there will herein be described in detail several specific embodiments with the understanding that the present disclosure is to be considered as an exemplification of the principles of the invention and is not intended to limit the invention to the embodiments illustrated.

In accordance with the present invention, a method for healing a wound is disclosed which comprises the steps of topically administering a wound healing composition to a wounded area, wherein the wound healing composition comprises: a first medicament characterized as a calcium channel blocker or pharmaceutically acceptable salts or solvates thereof; a second medicament characterized as a hemorrheologic agent or pharmaceutically acceptable salts or solvates thereof; and a dermal penetrating agent or pharmaceutically acceptable salts or solvates thereof.

It will be understood that regardless of it ordinary meaning the term “wounded area” will be defined herein as an area having an open and/or closed injury as well as any surrounding periphery that may, but not necessarily, involve a laceration or breaking of a membrane (e.g. skin) and may, but not necessarily, involve damage to underlying tissue. Indeed, in accordance with the present invention, many times a wound healing composition is associated with and/or applied to the surrounding periphery of the wound without actually applying the same to the open, closed, and/or irritated portion of the wound.

In accordance with the present invention, the first medicament comprises one or more calcium channel blocker(s), including, for example, dihydropyridines, phenylalkylamines, and benzothiazepines—just to name a few. Specific, non-limiting, examples include amlodipine, felodipine, nicardipine, nifedipine, nimodipine, nisoldipine, nitrendipine, lacidipine lercanidipine, verapamil, gallopamil, and diltiazem. It will be understood that the above-identified calcium channel blockers are readily available for any one of a number of common commercial sources.

In one aspect of the present invention, the first medicament (characterized as a calcium channel blocker) is preferably represented by the following chemical structure:

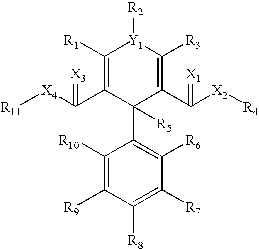

wherein R₁₋₁₁ are the same or different and comprise H, a hydroxy group, a primary, secondary, or tertiary amine, a halide, NO₂, SO₃H, CN, a straight or branched alkyl, cycloalkyl, polycycloalkyl, heterocycloalkyl, aryl, alkaryl, aralkyl, alkoxy, alkenyl, alkynyl, or carbonyl group containing approximately 1 to approximately 50 carbon atom(s), a silyl or siloxyl group containing approximately 1 to approximately 50 silicon atom(s), and combinations thereof, preferably with the proviso that at least one of R₆-R₁₀ comprises a halide, NO₂, SO₃H, or CN; wherein X₁₋₄ are the same or different and comprise oxygen, sulfur, or selenium; and wherein Y₁ comprises nitrogen or phosphorus. Preferably, the first medicament is represented by the following chemical structure:

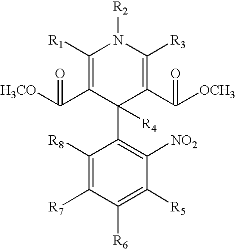

wherein R₁₋₈ are the same or different and comprise H, a hydroxy group, a primary, secondary, or tertiary amine, a straight or branched alkyl, cycloalkyl, polycycloalkyl, heterocycloalkyl, aryl, alkaryl, aralkyl, alkoxy, alkenyl, alkynyl, or carbonyl group containing approximately 1 to approximately 50 carbon atom(s), a silyl or siloxyl group containing approximately 1 to approximately 50 silicon atom(s), and combinations thereof. By way of a non-limiting specific example, the first medicament may comprise the following chemical structure:

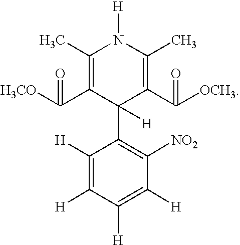

For purposes of clarity, and in an attempt to eliminate any potential ambiguity associated with the nomenclature of the above-identified medicament, it will be understood that a specific medicament provided herein above is defined as 3,5-pyridinedicarboxylic acid, 1,4-dihydro-2,6-dimethyl-4-(2-nitrophenyl)-dimethyl ester which is commercially available from Bayer Pharmaceuticals Corporation.

It will be understood that an “effective amount” of the above-identified first medicaments is an amount that facilitates healing of a wound, and can be administered, via any one of a number of conventional means, to a patient/subject. Preferably, the effective dose ranges in concentration from approximately 0.5% (wt.) to approximately 40% (wt.) approximately q.d.-q.i.d., and more preferably ranges in concentration from approximately 2% (wt.) to approximately 15% (wt.) approximately q.d.-q.i.d. However, the effective amount will vary depending upon many factors including, for example, the size and type of wound being treated.

For purposes of the present disclosure, the second medicament (characterized as a hemorrheologic agent) is preferably represented by the following chemical structure:

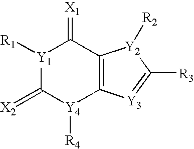

wherein R₁₋₄ are the same or different and comprise H, a hydroxy group, a primary, secondary, or tertiary amine, a straight or branched alkyl, cycloalkyl, polycycloalkyl, heterocycloalkyl, aryl, alkaryl, aralkyl, alkoxy, alkenyl, alkynyl, or carbonyl group containing approximately 1 to approximately 50 carbon atom(s), a silyl or siloxyl group containing approximately 1 to approximately 50 silicon atom(s), and combinations thereof; wherein X₁₋₂ are the same or different and comprise oxygen, sulfur, or selenium; and wherein Y₁₋₄ are the same or different and comprise nitrogen or phosphorus.
Preferably, the second medicament is represented by the following chemical structure:

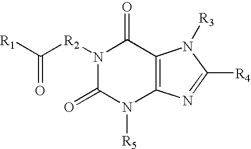

wherein R₁₋₅ are the same or different and comprise H, a hydroxy group, a primary, secondary, or tertiary amine, a straight or branched alkyl, cycloalkyl, polycycloalkyl, heterocycloalkyl, aryl, alkaryl, aralkyl, alkoxy, alkenyl, alkynyl, or carbonyl group containing approximately 1 to approximately 50 carbon atom(s), a silyl or siloxyl group containing approximately 1 to approximately 50 silicon atom(s), and combinations thereof. One specific example includes the following chemical structure:

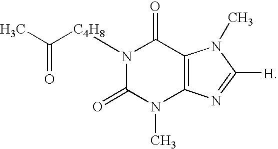

For purposes of clarity, and in an attempt to eliminate any potential ambiguity associated with the nomenclature of the above-identified second medicament, it will be understood that a specific medicament provided herein above is defined as 1-(5-oxohexyl)-3,7-dimethylxanthine, which is commercially available from Aventis Pharmaceuticals.

It will be understood that an “effective amount” of the above-identified second medicaments is an amount that facilitates healing of a wound, and can be administered, via any one of a number of conventional means, to a patient/subject. Preferably, the effective dose ranges in concentration from approximately 0.5% (wt.) to approximately 40% (wt.) approximately q.d.-q.i.d., and more preferably ranges in concentration from approximately 5% (wt.) to approximately 15% (wt.) approximately q.d.-q.i.d. However, the effective amount will vary depending upon many factors including, once again for example, the size and type of wound being treated.

In accordance with the present invention, the dermal penetrating agent is preferably represented by the following chemical structure:

R₁—X₁—R₂—X₂—R₃—X₃—R₄

wherein R₁₋₄ are the same or different and comprise H, a hydroxy group, a primary, secondary, or tertiary amine, a straight or branched alkyl, cycloalkyl, polycycloalkyl, heterocycloalkyl, aryl, alkaryl, aralkyl, alkoxy, alkenyl, alkynyl, or carbonyl group containing approximately 1 to approximately 50 carbon atom(s), a silyl or siloxyl group containing approximately 1 to approximately 50 silicon atom(s), and combinations thereof; and wherein X₁₋₃ are the same or different and comprise oxygen, sulfur, or selenium.

Preferably, the dermal penetrating agent is represented by the following chemical structure:

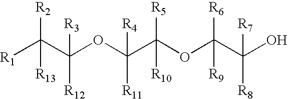

wherein R₁₋₁₃ are the same or different and comprise H, a hydroxy group, a primary, secondary, or tertiary amine, a straight or branched alkyl, cycloalkyl, polycycloalkyl, heterocycloalkyl, aryl, alkaryl, aralkyl, alkoxy, alkenyl, alkynyl, or carbonyl group containing approximately 1 to approximately 50 carbon atom(s), a silyl or siloxyl group containing approximately 1 to approximately 50 silicon atom(s), and combinations thereof. A specific example includes the following chemical structure:

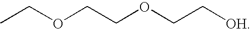

For purposes of clarity, and in an attempt to eliminate any potential ambiguity associated with the nomenclature of the above-identified dermal penetrating agents, it will be understood that a specific dermal penetrating agent provided herein above is defined as 2-(2-ethoxy-ethoxy)-ethanol, which is commercially available from Pfaltz and Bauer.

It will be understood that an “effective amount” of the above-identified dermal penetrating agents is an amount that facilitates healing of a wound, and can be administered, via any one of a number of conventional means, to a patient/subject. Preferably, the effective dose ranges in concentration from approximately 0.5% (wt.) to approximately 60% (wt.) approximately q.d.-q.i.d., and more preferably ranges in concentration from approximately 5% (wt.) to approximately 20% (wt.) approximately q.d.-q.i.d. However, the effective amount will vary depending upon many factors including, as mentioned herein, the size and type of wound being treated.

In support of the present invention, several experiments were conducted to evaluate the efficacy of above-identified first medicaments, second medicaments, and dermal penetrating agents in treating conventionally non-healing wounds. A topical formulation (80 mg/ml) was prepared comprising approximately 8% (wt.) of nifedipine (i.e. 3,5-pyridinedicarboxylic acid, 1,4-dihydro-2,6-dimethyl-4-(2-nitrophenyl)-dimethyl ester), 10% (wt.) pentoxifylline (i.e. 1-(5-oxohexyl)-3,7-dimethylxanthine), 12.5% (wt.) ethoxy diglycol (i.e. 2-(2-ethoxy-ethoxy)-ethanol), 22% (wt.) lecithin/isopropyl palmitate, and pluronic F-127 (to 100%). The above-identified composition was topically administered to the outer periphery of a plurality of subjects having a wounded area approximately q.d.-q.i.d., wherein it was verified that wounds which ordinarily were difficult or impossible to heal were, indeed, healed—and in some cases prevented amputation.

The foregoing description merely explains and illustrates the invention and the invention is not limited thereto except insofar as the appended claims are so limited, as those skilled in the art who have the disclosure before them will be able to make modifications without departing the scope of the invention.

What is claimed is:

1. A method for healing a wound, comprising the steps of:
topically administering a wound healing composition to a wounded area, wherein the wound healing composition comprises:
a first medicament characterized as a calcium channel blocker or pharmaceutically acceptable salts or solvates thereof;
a second medicament characterized as a hemorrheologic agent or pharmaceutically acceptable salts or solvates thereof; and
a dermal penetrating agent or pharmaceutically acceptable salts or solvates thereof.

2. The method for healing a wound according to claim 1, wherein the step of topically administering a wound healing composition comprises the step of topically administering an effective amount of a wound healing composition to the wounded area.

3. The method for healing a wound according to claim 1, wherein the step of topically administering a wound healing composition comprises the step of topically administering a wound healing composition which comprises a first medicament represented by the following chemical structure: 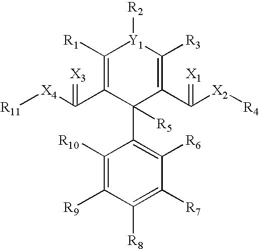
wherein R₁₋₁₁ are the same or different and comprise H, a hydroxy group, a primary, secondary, or tertiary amine, a halide, NO₂, SO₃H, CN, a straight or branched alkyl, cycloalkyl, polycycloalkyl, heterocycloalkyl, aryl, alkaryl, aralkyl, alkoxy, alkenyl, alkynyl, or carbonyl group containing approximately 1 to approximately 50 carbon atom(s), a silyl or siloxyl group containing approximately 1 to approximately 50 silicon atom(s), and combinations thereof, wherein X₁₋₄ are the same or different and comprise oxygen, sulfur, or selenium; and wherein Y₁ comprises nitrogen or phosphorus.

4. The method for healing a wound according to claim 1, wherein the step of topically administering a wound healing composition comprises the step of topically administering a wound healing composition which comprises a first medicament represented by the following chemical structure: 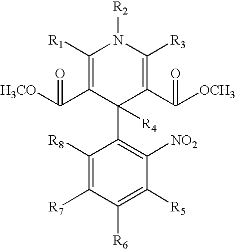
wherein R₁₋₈ are the same or different and comprise H, a hydroxy group, a primary, secondary, or tertiary amine, a straight or branched alkyl, cycloalkyl, polycycloalkyl, heterocycloalkyl, aryl, alkaryl, aralkyl, alkoxy, alkenyl, alkynyl, or carbonyl group containing approximately 1 to approximately 50 carbon atom(s), a silyl or siloxyl group containing approximately 1 to approximately 50 silicon atom(s), and combinations thereof.

5. The method for healing a wound according to claim 4, wherein the step of topically administering a wound healing composition comprises the step of topically administering an effective amount of a wound healing composition to the wounded area.

6. The method for healing a wound according to claim 1, wherein the step of topically administering a wound healing composition comprises the step of topically administering a wound healing composition which comprises a first medicament represented by the following chemical structure:

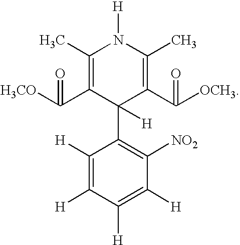

7. The method for healing a wound according to claim 6, wherein the step of topically administering a wound healing composition comprises the step of topically administering an effective amount of a wound healing composition to the wounded area.

8. The method for healing a wound according to claim 1, wherein the step of topically administering a wound healing composition comprises the step of topically administering a wound healing composition which comprises a second medicament represented by the following chemical structure: 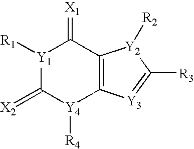
wherein R₁₋₄ are the same or different and comprise H, a hydroxy group, a primary, secondary, or tertiary amine, a straight or branched alkyl, cycloalkyl, polycycloalkyl, heterocycloalkyl, aryl, alkaryl, aralkyl, alkoxy, alkenyl, alkynyl, or carbonyl group containing approximately 1 to approximately 50 carbon atom(s), a silyl or siloxyl group containing approximately 1 to approximately 50 silicon atom(s), and combinations thereof; wherein X₁₋₂ are the same or different and comprise oxygen, sulfur, or selenium; and wherein Y₁₋₄ are the same or different and comprise nitrogen or phosphorus.

9. The method for healing a wound according to claim 1, wherein the step of topically administering a wound healing composition comprises the step of topically administering a wound healing composition which comprises a second medicament represented by the following chemical structure: 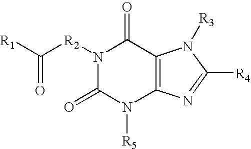
wherein R₁₋₅ are the same or different and comprise H, a hydroxy group, a primary, secondary, or tertiary amine, a straight or branched alkyl, cycloalkyl, polycycloalkyl, heterocycloalkyl, aryl, alkaryl, aralkyl, alkoxy, alkenyl, alkynyl, or carbonyl group containing approximately 1 to approximately 50 carbon atom(s), a silyl or siloxyl group containing approximately 1 to approximately 50 silicon atom(s), and combinations thereof.

10. The method for healing a wound according to claim 9, wherein the step of topically administering a wound healing composition comprises the step of topically administering an effective amount of a wound healing composition to the wounded area.

11. The method for healing a wound according to claim 1, wherein the step of topically administering a wound healing composition comprises the step of topically administering a wound healing composition which comprises a second medicament represented by the following chemical structure:

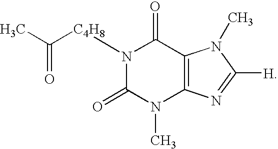

12. The method for healing a wound according to claim 11, wherein the step of topically administering a wound healing composition comprises the step of topically administering an effective amount of a wound healing composition to the wounded area.

13. The method for healing a wound according to claim 1, wherein the step of topically administering a wound healing composition comprises the step of topically administering a wound healing composition which comprises a dermal penetrating agent represented by the following chemical structure:
R₁—X₁—R₂—X₂—R₃—X₃—R₄
wherein R₁₋₄ are the same or different and comprise H, a hydroxy group, a primary, secondary, or tertiary amine, a straight or branched alkyl, cycloalkyl, polycycloalkyl, heterocycloalkyl, aryl, alkaryl, aralkyl, alkoxy, alkenyl, alkynyl, or carbonyl group containing approximately 1 to approximately 50 carbon atom(s), a silyl or siloxyl group containing approximately 1 to approximately 50 silicon atom(s), and combinations thereof; and wherein X₁₋₃ are the same or different and comprise oxygen, sulfur, or selenium.

14. The method for healing a wound according to claim 1, wherein the step of topically administering a wound healing composition comprises the step of topically administering a wound healing composition which comprises a dermal penetrating agent represented by the following chemical structure: 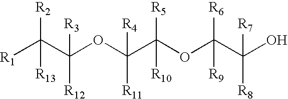
wherein R₁₋₁₃ are the same or different and comprise H, a hydroxy group, a primary, secondary, or tertiary amine, a straight or branched alkyl, cycloalkyl, polycycloalkyl, heterocycloalkyl, aryl, alkaryl, aralkyl, alkoxy, alkenyl, alkynyl, or carbonyl group containing approximately 1 to approximately 50 carbon atom(s), a silyl or siloxyl group containing approximately 1 to approximately 50 silicon atom(s), and combinations thereof.

15. The method for healing a wound according to claim 14, wherein the step of topically administering a wound healing composition comprises the step of topically administering an effective amount of a wound healing composition to the wounded area.

16. The method for healing a wound according to claim 1, wherein the step of topically administering a wound healing composition comprises the step of topically administering a wound healing composition which comprises a dermal penetrating agent represented by the following chemical structure:

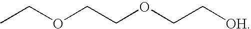

17. The method for healing a wound according to claim 1, wherein the step of topically administering a wound healing composition comprises the step of topically administering a wound healing composition which comprises a first medicament represented by the following chemical structure: 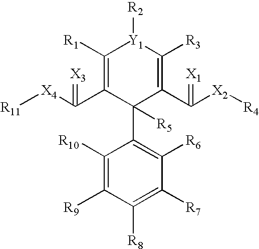
wherein R₁₋₁₁ are the same or different and comprise H, a hydroxy group, a primary, secondary, or tertiary amine, a halide, NO₂, SO₃H, CN, a straight or branched alkyl, cycloalkyl, polycycloalkyl, heterocycloalkyl, aryl, alkaryl, aralkyl, alkoxy, alkenyl, alkynyl, or carbonyl group containing approximately 1 to approximately 50 carbon atom(s), a silyl or siloxyl group containing approximately 1 to approximately 50 silicon atom(s), and combinations thereof, wherein X₁₋₄ are the same or different and comprise oxygen, sulfur, or selenium; and wherein Y₁ comprises nitrogen or phosphorus; a second medicament represented by the following chemical structure: 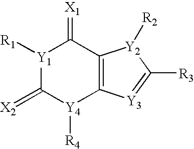
wherein R₁₋₄ are the same or different and comprise H, a hydroxy group, a primary, secondary, or tertiary amine, a straight or branched alkyl, cycloalkyl, polycycloalkyl, heterocycloalkyl, aryl, alkaryl, aralkyl, alkoxy, alkenyl, alkynyl, or carbonyl group containing approximately 1 to approximately 50 carbon atom(s), a silyl or siloxyl group containing approximately 1 to approximately 50 silicon atom(s), and combinations thereof; wherein X₁₋₂ are the same or different and comprise oxygen, sulfur, or selenium; and wherein Y₁₋₄ are the same or different and comprise nitrogen or phosphorus; and a dermal penetrating agent represented by the following chemical structure:
R₁—X₁—R₂—X₂—R₃—X₃—R₄
wherein R₁₋₄ are the same or different and comprise H, a hydroxy group, a primary, secondary, or tertiary amine, a straight or branched alkyl, cycloalkyl, polycycloalkyl, heterocycloalkyl, aryl, alkaryl, aralkyl, alkoxy, alkenyl, alkynyl, or carbonyl group containing approximately 1 to approximately 50 carbon atom(s), a silyl or siloxyl group containing approximately 1 to approximately 50 silicon atom(s), and combinations thereof; and wherein X₁₋₃ are the same or different and comprise oxygen, sulfur, or selenium.

18. The method for healing a wound according to claim 17, wherein the step of topically administering a wound healing composition comprises the step of topically administering an effective amount of a wound healing composition to the wounded area.

19. The method for healing a wound according to claim 1, wherein the step of topically administering a wound healing composition comprises the step of topically administering a wound healing composition which comprises a first medicament comprising 3,5-pyridinedicarboxylic acid, 1,4-dihydro-2,6-dimethyl-4-(2-nitrophenyl)-dimethyl ester and pharmaceutically acceptable salts or solvates thereof; a second medicament comprising 1-(5-oxohexyl)-3,7-dimethylxanthine and pharmaceutically acceptable salts or solvates thereof; and a dermal penetrating agent comprising 2-(2-ethoxy-ethoxy)-ethanol and pharmaceutically acceptable salts or solvates thereof.

20. The method for healing a wound according to claim 19, wherein the step of topically administering a wound healing composition comprises the step of topically administering an effective amount of a wound healing composition to the wounded area.
